(19) United States
(12) Patent Application Publication
(10) Pub. No.: US 2004/0258729 A1
(43) Pub. Date: Dec. 23, 2004

(54) Tissue engineering scaffolds

(76) Inventors: Jan Tadeusz Czernuszka, Oxford (GB); Eleftherios Sachlos, Oxford (GB); Brian Derby, Manchester (GB); Nuno A.E. Reis, Lisbon (PT); Christopher Charles Ainsley, Exeter (GB)

Correspondence Address: QUARLES & BRADY LLP, 411 E. WISCONSIN AVENUE, SUITE 2040, MILWAUKEE, WI, 53202-4497, US

(21) Appl. No.: 10/489,295

(22) PCT Filed: Mar. 11, 2002
(86) PCT No.: PCT/GB02/04139

(30) Foreign Application Priority Data
Sep. 11, 2001 (GB) 0121985.6

(51) Int. Cl. A61F 2/00

(52) U.S. Cl. 424/426

(57) ABSTRACT

A process for preparing a scaffold of biocompatible polymer which comprises placing a composition comprising the polymer in a mould possessing one or more voids therein, said mould being a negative of the desired shape including a designed architecture and dimensions of the scaffold, causing the polymer to acquire the shape of the mould and causing pores to be formed in the polymer, and removing the mould without affecting the polymer.

This invention relates to tissue engineering scaffolds.

Tissue engineering is a new multidisciplinary field that involves the development of biological substitutes that restore, maintain or improve tissue function. This field has the potential of overcoming the limitations of conventional treatments by producing a supply of organ and tissue substitutes biologically tailored to the patient.

Tissue engineering involves growing the relevant cell(s) in the laboratory into the required organ or tissue. However, unaided cells lack the ability to grow in favoured orientations and thus define the anatomical shape of the organ and tissue. Instead, they randomly migrate to form a two dimensional layer of cells. Thus, three dimensional (3D) tissues are required and this is achieved by the use of 3D scaffolds, which act as substrates for cellular attachment. Scaffolds are required to 1) have porosity, generally interconnecting, so as to allow tissue integration and blood vessel colonisation, 2) be made of a biodegradable or bioresorbable material so that tissue can eventually replace the scaffold as it degrades, 3) have appropriate surface chemistry to favour cell attachment, proliferation and differentiation, 4) possess adequate mechanical properties to match the intended implantation site and 5) be easily fabricated into a variety of shapes and sizes. In particular, the pore size of the scaffold has been identified as critical for the successful growth of tissues. An average pore size range of 200 to 400 μm has been shown as optimum for the growth of bone tissue.

Biodegradable and bioresorbable polymers and ceramics have been used as the material to make the scaffolds. The majority of the work has focussed on polymers since ceramic scaffolds have been aimed mostly at bone tissue engineering. The polymers which have been used are synthetic (e.g. polylactic acid and polyglycolic acid, FDA approved polymers used for sutures and orthopaedic fixation screws), or natural (e.g. collagen, an abundant protein present in the connective tissue of mammals which is FDA approved—the collagen can be from cow hide and used to correct skin contour defects).

Several techniques have been developed to produce tissue engineering scaffolds from biodegradable and bioresorbable polymers. For synthetic polymers, these are usually based on solvent casting-particulate leaching, phase separation, gas foaming and fibre meshes. For natural collagen scaffolds, these can be made by freezing a dispersion/solution of collagen and then freeze-drying it. Freezing the dispersion/solution results in the production of ice crystals that grow and force the collagen into the interstitial spaces, thus aggregating the collagen. The ice crystals are removed by freeze-drying which involves inducing the sublimation of the ice and this gives rise to pore formation; therefore the water passes from a solid phase directly to a gaseous phase and eliminates any surface tension forces that can collapse the delicate porous structure. These techniques are, however, generally dependent on a pore generator to form the pores within the scaffold, e.g. salt particles, liquid-liquid phase separation, gas bubble evolution or ice crystals. However, the distribution of pores and fibre bonding locations cannot be precisely controlled and consequently these techniques are unable to ensure reliable interconnection and distribution of pores within the scaffold. Consequently, these techniques cannot produce complicated internal features, like channels, that can act as an artificial vascular system which would favour the growth of blood vessels and could sustain the cell growth deep into the scaffold. In this connection it should be borne in mind that as a general rule the parenchymal or supportive cells of vascularised tissues in vivo (except cartilage) are no further than 25-50 μm from the nearest blood vessel.

Solid Freeform Fabrication (SFF) (also known under the generic name of Rapid Prototyping (RP)) technologies have the potential to significantly impact on tissue engineering by producing scaffolds with tailored architectures and thus overcome the limitations of the current fabrication techniques. SFF processes involve producing three-dimensional objects directly from a computer-aided design model using layered manufacturing strategies. They are capable of delivering complex shapes exhibiting intricate internal features directly from computer-generated models.

According to the present invention there is provided a process for preparing a scaffold of polymer, generally a biocompatible polymer, ideally biodegradable or bioresorbable in nature for tissue engineering purposes, which comprises placing a composition comprising the polymer in mould possessing one or more voids therein, said mould being a negative of the desired shape of the scaffold, causing the polymer to acquire the shape of the mould, removing the mould and causing pores to be formed in the polymer, and without affecting the polymer.

The process of the present invention is particularly applicable to making scaffolds of collagen but it is also applicable to other naturally occurring polymers and proteins including elastin, fibrin, albumen, silk, gelatin and proteoglycans like hyaluronic acid, chondroitin sulfate, dermatan sulfate, keratan sulfate and chitin as well as mixtures, in particular a mixture of collagen and elastin which can, if desired, subsequently be crosslinked. The present invention can also be applied to synthetic biodegradable and bioresorbable polymers including polylactic acid and polyglycolic acid well as polyethyleneglycol-polyester and ethylene oxide-polyester copolymers. These polymers can be used alone or together with, for example, a bioceramic to make a composite scaffold. Bone is a composite structure that is made up of a collagen matrix, reinforced with hydroxyapatite (HA) crystals. Scaffolds, which resemble the chemical composition of bone can be produced by mixing HA particles. The weight ratio of HA/collagen in human bone is about 2:1 so that the collagen used should desirably be mixed with HA in roughly this ratio. This ratio can, of course, be varied by adjusting the amount of HA incorporated.

Obtaining a mould made from sacrificial material is more important than how to make the mould although it will of course be appreciated that if the mould is to be of any value as a negative it should not generally be porous. For this reason several techniques can be used to make the moulds including injection moulding, computerised numerical control milling and solid freeform fabrication (SFF) just to name a few. It is a particular feature of the process of the present invention that the mould which acts as a sacrificial member can be made using SFF technologies including three dimensional printing, ballistic particle manufacturing, fusion deposition modelling, selective laser sintering and stereo-lithography but preferably phase change jet printing. Accordingly, the mould can have an intricate shape which is desirable for the resulting scaffold, including for example, channels and pores, and for this reason SFF is the technology of choice for the invention.

In a preferred embodiment, the mould is produced with the negative shape of the scaffold using phase change jet printing strategies. One such system is known under the mark Model Maker II (Solidscape Inc, Merrimak, N.H., USA).

The system comprises two ink-jet print-heads, each delivering a different material, one material for building the actual mould and the other acting as support for any unconnected or overhanging features. Molten microdroplets are generated by the jet heads which are heated above the melting temperature of the material, and deposited in a drop-on-demand fashion. The microdroplets solidify on impact, cooling to form a bead. Overlapping of adjacent beads forms a line, overlapping of adjacent lines forms a layer. Each layer is deposited by repeated sweep deposition of continuous beads on a vector mode operation basis. After solidification, a horizontal rotary cutter can be used to flatten the top surface of a recently deposited layer and control the thickness. The platform is lowered and the process is repeated to build the next layer, which adheres to the previous, until the shape of the mould is completed. Once built, the mould can then be immersed in a selective solvent for the support structure but a non-solvent for the build material and leave the physical mould in its desired shape which is the principle behind the commercial system. The removal of support material from the mould can also be based on a one solvent system, but the support and mould material must have different rates of dissolution in the solvent i.e. the support dissolves away faster than the mould material.

Typically, the build material is a polar material and the support material is non polar so that the support material can be removed by immersion of the mould in a non-polar solvent (or vice versa). It can also be possible to use a system where both the support material and the mould material are dissolved by the same solvent but the rates of dissolution are different such that the support is dissolved away before any mould material is dissolved. Typically, therefore, the support material is wax or other similar material with a viscosity typically of 5 to 40 centipoises at the printing temperature, for example a non-polar wax such as candelilla wax, optionally with a fatty ester such as N2-hydroxyethyl stearamide. It will be appreciated that the mould material and the support material should have similar melting points and similar thermal coefficients of expansion. The build material is, in this instance, typically a polar resin such as a polyester resin, for example a linear, saturated polyester, typically a copolymer produced by condensation polymerisation of one or more glycols and one or more dibasic acids or esters. If desired the polar resin can be extended with a filler which itself should, of course, be polar. Typical fillers which can be used for this purpose include sulphonamides, typically aromatic sulphonamides and, especially o-and p-toluene sulphonamides since these possess a melting point similar to that of the resin.

The mould material can be a biocompatible polymer that is optionally biodegradable but should be soluble in a solvent such as ethanol, amyl acetate or propanone as these can be used with the critical point dryer, as discussed below. Suitable biocompatible materials which can be used for this purpose and which possess the properties for printing with the ink jet printer include cholesterol, which is preferred, phosphatidyl choline and other lipid—or lipoprotein-based molecules.

A candidate support material is polyethylene glycol (PEG). This biocompatible and biodegradable polymer possesses the properties which enable it to be printed by the ink jet printing system and is soluble in water but insoluble in ethanol. Other support materials include those which are soluble in water and insoluble in ethanol, amyl acetate or propanone. Candidate support materials which are biocompatible and possess the properties for printing with the ink jet printer include polyethylene oxide (PEO), polyvinyl alcohol (PVA) and L-malic acid. Again, biocompatibility is desirable for the reasons given above.

PEG could also be used as the mould material. However, in this system a solution of water and crosslinking agents would be required, firstly to dissolve the mould and secondly to induce crosslink formation in the collagen. Critical point drying would not be required in this instance.

A particular combination of build and support materials which can be used are those sold under the marks ProtoBuild and ProtoSupport, respectively by Solidscope Inc. The selective solvent for ProtoSupport is the proprietary BioAct. The build material is believed to have the following composition:

| Formula I | | Parts by weight |
|---|---|---|
| a) | Ketjenflex 9S | 90 |
| b) | Vitel 5833 | 10 |
| c) | Ultranox 626 | 1 |
| or | | |
| Formula 2 | | |
| a) | Ketjenflex 9S | 85 |
| b) | Vitel 5833 | 10 |
| c) | Ultranox 626 | 1 |
| d) | Iconol NP-100 | 5 |

where
a) Ketjenflex 9S is 40/60 blend of ortho-toluene sulfonamide/para-toluene sulfonamide, available from Akzo Chemie - Chicago, Illinois.
b) Vitel 5833 is a polyester resin available from Shell Chemical Company - Akron, Ohio.
c) Ultranox 626 is a phosphite antioxidant available from G.E. Specialty Chemicals Inc. - Parkersburg, West Virginia.
d) Iconol NP-100 is a nonylphenol ethoxylate available from BASF Performance Chemicals - Parsippany, New Jersey.

The support material is believed to have the following composition:

| | | Parts by weight |
|---|---|---|
| a) | Candelilla Wax | 65 |
| | Refined, light flakes | |
| b) | CPH-380-N | 20 |
| c) | Ross Wax 100 | 10 |
| d) | Eastotac - H 130 | 5 |
| | or | |
| | H 100 | |
| e) | Irganox 1010 | 2 |

where:
a) Candelilla Wax a is low resin natural wax available from Frank B. Ross Co., Inc. - Jersey City, New Jersey.
b) CPH-380-N is N,2-hydroxyethyl stearamide available from the C.P. Hall Company - Chicago, Illinois.
c) Ross Wax 100 is Fischer-Tropsch Wax available from Frank B. Ross Co.
d) Estotac is H 130 or H 100 - Hydrocarbon resin available from Eastman Chemical Products, Inc. - Kingsport, Tennessee.
e) Irganox 1010 is a hindered phenol antioxidant available from Ciba - Geigy Additives - Hawthorne, New York.

Once the mould has been made and the support material removed, it is ready to receive the composition comprising the biocompatible, preferably biodegradable or bioresorbable, polymer which is to form the scaffold. As indicated above, collagen is the preferred material and the subsequent description will refer to this although it will be appreciated that the other biocompatible polymers mentioned above can be used in a similar way. Collagen not only serves as a structural component in many tissues but also as a chemotactic (cell-attracting) agent for several cell types. Therefore collagen exhibits enhanced cellular attachment and provides an environment that resembles more the natural extra-cellular matrix of the tissue compared to synthetic polymers.

A solution or dispersion of collagen can be used to cast in the mould. The concentration of collagen is desirably as high as possible. Usually, a dispersion of the collagen in water is used, typically, with a concentration of the dispersion is from 0.01 to 10% or more, more particularly 0.1 or 0.5 to 5% and especially 0.75 to 2%, weight/volume. The viscosity of the dispersion increases with an increase in the concentration of collagen. Therefore, highly concentrated collagen dispersions possess a high viscosity and are unable to easily flow into small features of the mould. This results in a trade-off between maximising the amount of collagen in the mould and ensuring that the collagen flows into all the fine features of the mould. This complication can be overcome by casting a low viscosity dispersion of collagen into the mould and then inserting a removable absorbent for the liquid such as chromatographic paper into the collagen dispersion. The concentration of collagen in the mould is increased because the paper effectively sucks up the water component of the dispersion. Repeated steps of casting and paper chromatography treatment are usually required to maximise the concentration of collagen in the mould before freezing. The nature of the collagen is not particularly critical. Thus it can be type I collagen as present in bone, skin, tendon, ligaments, cornea and internal organs or type II collagen which is present in cartilage, invertebral disk, notochord and the vitreous humour of the eye. More than 15 collagen types have been discovered in varying concentrations in different tissues and more are likely to be discovered in the future. The use of bovine collagen is particularly convenient as it is abundant. However, other sources like recombinant human collagen from transgenic animals are attractive for this application.

The presence of a weak acid such as acetic acid in the collagen dispersion causes a reduction in the pH to a level which can be slightly below that at which collagen starts to swell and dissolve. This can facilitate the formation of the dispersion. The composition can be cast in the mould.

The extracellular matrix can be made up of collagen. However other proteins like elastin, and glycoaminoglycans like chondroitin sulphate, dermatan sulphate, hyaluronic acid, heparin sulphate and keratin sulphate can also be present. The percent composition of these other proteins and glycoaminoglycans, and their spatial distribution, along with the appropriate collagen type constitute an extracellular matrix that is specific for a particular tissue type. For example, in the aorta artery there is approximately 39% collagen and 24% elastin; this same percentage can also be achieved according to the present invention by mixing the appropriate ratio of elastin, and any other relevant molecules, with the collagen dispersion to produce a scaffold that resembles the chemical composition of the extracellular matrix of the aorta artery.

Collagen is the major protein constituent of the extracellular matrix of human tissue and is therefore an important scaffold component. However, it is appreciated that scaffolds without collagen may be required. This can be achieved by using a biological relevant casting fluid other than collagen. A biologically relevant fluid, as used herein, means any molecule which can effectively act as an extracellular matrix and is able to support or induce the attachment, migration, proliferation, differentiation and survival of the favoured cell types, as well as suppressing the unfavoured cell types, being cultured. Thus the casting fluid or liquid does not necessarily have to contain collagen. Other proteins, specifically extracellular matrix proteins, and glycoaminoglycans can also be used. Solutions or dispersions based on, for example, elastin, hyaluronic acid, aggrecan, chitosan, vegetable gel, starch and agar can be formulated and used either on their own or in combination with each other to make the required scaffold.

A number of biologically relevant molecules which can regulate the gene activity of the cultured cells can be added to collagen dispersions whilst in the liquid phase. For example, bioactive ceramic particles like hydroxyapatite or Bioglass™, biochemical nucleators for the precipitation of calcium phosphate like phosphoserine and other biochemicals with an affinity to bind calcium, glycoaminoglycans, proteoglycans, polysaccharides, hormones and growth factors, enzymes, nucleic acids, lipids, extracellular matrix proteins like elastin, fibronectin and laminin and synthetic biodegradable polymers can also be used, generally in combination with collagen. Antibiotics can be incorporated to prevent infection of the cultured tissue and the site of implantation. Immunosuppressant drugs can also be incorporated to reduce any possible rejection reaction associated with cultured cells that may be ‘foreign’ or of an allogenic nature to the recipient patient. Surfactants, which can increase the castability of the dispersion formulation into the mould, can also be incorporated.

As indicated above, after the collagen composition has been placed in the mould it is generally frozen so as to force the collagen into the interstitial spaces. In accordance with a preferred embodiment, in the process of the present invention the dispersion is first frozen, typically for about 24 hours and then the mould is removed. The rate at which the dispersion is frozen and the pH have an effect on the resulting pore size. As is known the faster the dispersion is frozen, the smaller the resulting pores will be. Typically the temperature of freezing is from −20° C. for larger pores to −80° C. for the smallest pores, but the size can of course be controlled by adjusting the rate of cooling. This technique allows control over the micropores i.e. the pores created by the ice crystals. However, pores of any shape can also be created by making the mould with the required negative shape e.g. connecting spheres running across the mould will produce well defined spherical pores. For other polymers, there is the option of inducing polymerisation of the monomer or crosslinking the polymer after casting into the mould.

The orientation of the collagen molecules is important in relation to the quality of the cultured tissue. For example, the collagen fibres in skin are orientated randomly whereas during wound healing of the skin the fibres become orientated more in parallel to produce poorly aesthetic scar tissue. The natural magnetic and electrical properties of collagen can be used to orientate the fibres appropriately. This can be achieved by casting the collagen solution or dispersion in the mould and using appropriately placed electrodes in the mould to apply an electrical field or using an appropriately orientated magnet to produce a magnetic field in the favoured direction and allowing time for the collagen fibres to reorganise themselves before freezing. The same desired effect can be achieved by grafting electrical or magnetic particles, preferably of nanoscale dimensions, onto the collagen and then applying the electric or magnet field.

Electrical or magnetic particles, preferably nanoparticles, can also be incorporated into, or coated onto, the mould. The natural electrical and magnetic properties of collagen can then orientate the fibres appropriately. Such electrical or magnetic particles can be grafted onto the collagen and electrical particles of opposite charge then incorporated on the surface of the mould forcing the collagen to orientate along the mould, or repelling the collagen by using particles of the same charge. The same effect can be achieved by using magnetic particles. It will be appreciated that these electrical and magnetic particles can be incorporated into the mould material before the mould is made; the drop-on-demand control offered by ink-jet printing allows on to control the exact location and distribution of these particles.

Freezing can also be used to orientate the collagen fibres. By controlling the direction and rate of freezing the ice crystals that are formed can be used to push the collagen into the favoured orientation. The mould can be made of different materials, each with a different thermal conductivity which create, thermal gradients that allows the ice crystals to grow in the favoured direction. It will be appreciated that the ability to use multiple jet heads with the ink jet printing system allows for the delivery of such different materials to a predefined location.

The spatial distribution of the dispersion can also be controlled to produce chemically distinct regions within the scaffold that favour the growth of different tissue types. For example, the human joint contains bone, cartilage, ligament, tendon and synovial capsule tissue. Each of these tissues contain a chemically unique extracellular matrix. Laminated or mosaic structures, where each laminate or mosaic unit is chemically distinct, can be created by using a series of casting and freezing steps. For example, collagen can be cast into a mould and frozen, then elastin cast and frozen and the process repeated to produce a collagen-elastin composite which can then be dehydrated in ethanol and critical point dried.

Next the mould has to be removed. As indicated this must be done in a way which does not adversely affect the polymer. Thus it will be appreciated that it is not possible to use too much heat, as in firing, for this purpose since this would cause the collagen to denature or degrade. Rather, it is preferred to dissolve the mould away using a non-solvent for collagen, generally whilst being kept below 25° C. Collagen is generally stable at a pH of 4 to 10 so that if the mould material is sensitive to weak acid or weak alkali then such solutions can be used to dissolve away the mould. Alternatively, a hydrolysable salt can be used to make the mould and this can be eliminated after the scaffold has formed by the addition of the appropriate hydrolysate.

It is, however, preferred that the mould is removed by the use of a polar solvent since collagen is unaffected by it; in particular, one can use water, a ketone, an ester or an alcohol, especially one with 1 to 6 carbon atoms such as ethanol or 2-propanol or propanone, aryl acetate or an aqueous solution of such a solvent e.g. an aqueous ethanolic solution. Clearly, it is desirable to use a solvent which does not adversely affect human cells in any way in case of any residues while quickly dissolving the mould and for this purpose ethanol is preferred.

The procedure can be varied, in numerous ways, for example as follows:

Cast collagen in the mould and freeze, use water to dissolve the mould e.g. of PEG, then dehydrate by immersing in ethanol and critical point dry.

Cast collagen in the mould, add crosslinking agent to the collagen, allow time for crosslinks to form and then freeze, dehydrate in ethanol and critical point dry.

Cast collagen in the mould and freeze, use a solution of water and crosslinking agent to dissolve the mould and induce crosslink formation. Wash collagen scaffold with water to remove excess crosslinking agent.

The collagen scaffold which remains is generally in the form of a sponge-like material. Freeze-drying a frozen collagen dispersion, which involves removing the ice crystals by sublimation, produces a sponge with interconnecting porosity. Immersing a frozen dispersion of collagen in a (polar) non-solvent dissolves the ice crystals and produces a sponge-like structure similar to that obtained by freeze-drying, the major difference being that the collagen sponge is now suspended in the non-solvent. Furthermore, the non-solvent may be inducing stiffness to the collagen fibrils by dehydrating them. If water is not used, removal of the solvent is crucial. Critical point drying with liquid carbon dioxide can be used for this purpose. The solvent can also be removed by exchanging it with water. In this instance, the collagen sponge does not require critical point drying, and may be used for the subsequent stages of crosslinking and cell culturing, or an intermediate step of freezing the substituted water and freeze-drying the collagen may be incorporated to facilitate crosslinking before cell culturing. It will be appreciated that removal of the solvent by air-drying is generally not appropriate as the surface tension forces created during evaporation result in a collapse of the delicate porous structure one is trying to create.

According to a preferred embodiment, the article is in the non-solvent and subjected to critical point drying. This is a known technique whereby the article is placed in a pressurised container at, for example, 50 bars pressure with liquid carbon dioxide. The alcohol which is the more dense goes to the base of the container and is replaced by the CO₂. Thus it is possible to remove the solvent within the collagen by substituting it with liquid carbon dioxide. If one then increases the temperature from, say, 15-20° C. to e.g. 33-36° C. with a consequent increase in pressure (to 90 bars) the liquid carbon dioxide will gasify and escape. This results in a dry scaffold which is inherently porous and which retains the internal features dictated by the mould. The dry collagen scaffold can then, if desired, be crosslinked to increase the mechanical strength, decrease the antigenicity and decrease the degradation rate of the scaffold. Crosslinking can be accomplished by both physical and chemical techniques. Physical crosslinking can be achieved by dehydrothermal treatment and UV or gamma irradiation. Aldehydes such as glutaraldehyde and formaldehyde, polyepoxy resin, acyl azides, carbodiimides and hexamethylene compounds can be used for chemical crosslinking.

By means of the process of the present invention it is possible to obtain a collagen scaffold which has channels within it which are sufficiently close to one another to favour tissue growth. In the human body no cell (except cartilage) exists further than 25-50 μm from a blood vessel. Accordingly, it is desirable that in the scaffold there is never a distance greater than 50-100 μm between voids. Spheres as well as channels can be constructed using the requisite mould shape. Naturally, the degree of fineness of the structure is determined by the resolution of the equipment making the mould but resolutions as little as 150 μm are already achievable. Due to collagen's abundance in many tissues of the human body it should be appreciated that these collagen scaffolds could be used to grow most types of tissue.

In order to minimise the possible risk of contamination to the resultant scaffold in use it is preferred that the mould is made from a biocompatible material itself such that the scaffold does not cause any adverse response when implanted into the human body.

In general, it has been found that the critical point drying procedure results in some shrinkage of the scaffold but this can in fact be advantageous since it enables one to obtain somewhat smaller pores then can be resolved by the equipment. Thus it is possible to start with a mould which is somewhat larger than desired.

After rehydration and optional crosslinking the scaffolds are ready for cell culturing. For this purpose a continuous or peristaltic pump can be connected to the channels of the scaffold and a liquid which chemically favours or accelerates the attachment, proliferation, migration, differentiation and/or survival of cell types, and/or suppresses unfavoured cell types which chemically resembles human blood is forced to flow through the channel. In addition, a series of microsyringes can be inserted into the scaffold at exact locations that allow the deliverance of growth factors at time controlled periods. This allows for the spatial and chemical control of growth factors during favoured time periods. A combination of extracellular matrix and culture medium is generally required to produce a microenvironment favourable for the growth of cells. The scaffold provides the extracellular matrix requirement and the flow of a liquid medium rich in biochemicals which favours or accelerates the attachment, proliferation, migration, differentiation and survival of the respective cell types, as well as suppressing the growth of unfavoured cell types, through the channels of the scaffolds provides the vital signals required for the culturing of tissue. It will be appreciated that different cell types possess differences in cellular metabolic requirements and therefore the composition of the liquid medium is highly specific for each cell type. The medium should contain certain essential molecules such as oxygen, carbon dioxide, glucose, amino acids, albumin, globulin, fibrogen, cholesterol, phospholipids, triglycerids, minerals, trace elements and electrolytes e.g. cations of sodium, potassium, calcium, magnesium and anions e.g. chlorine, bicarbonate, phosphate and sulphate, vitamins, growth factors and hormones. It may also be advantageous to incorporate red and white blood cells to transport some of the above mentioned molecules and assist in the defence system of the scaffold. The purpose of the liquid medium flowing through the channels of the scaffold is to effectively act as an artificial vascular system which can support and sustain the growth of cells throughout the whole scaffold.

It will be appreciated that the scaffolds are readily reproducible and can act as a vehicle for research into the exact condition that favours tissue growth. The scaffolds can take the form of conduits, for example to support axonal growth of peripheral nerves, and/or to produce (grow) blood vessels, connective tissues like bone, cartilage, ligament, muscle and highly vascularised vital organs like heart, lung, liver, pancreas and kidney, and/or for the provision of nutrients for such growth.

The scaffolds of the present invention also find utility in bone formation, for example using the procedure described in 13^{th} European Conference on Biomaterials, Goteburg, Sweden, 4-7 September 1997 and A C Lawson, D. Phil Dissertation, University of Oxford, 1998.

It will also be appreciated that the external shape of the scaffold can be controlled. This is done by giving the walls of the mould the shape required. This means that one can make the gross shape of the organ, e.g. a cylinder for a long bone, or bean-shaped to make a kidney. Thus medical scans can be used to customise the shape of the external scaffold. For example taking an accident patient who has severe maxillo-facial traumas on the left side of his face, a Computerised Tomography (CT) or Magnetic Resonance Imaging (MRI) scan of the face can be taken. These scans produce two-dimensional (2D) slices of the volume that is scanned. Using computer software, the 2D slices can be stacked on top of each other to produce a virtual 3D image of the patient's skull showing the fractured region on the left hand side. Using more software functions the fractured region or defect can be corrected based on the symmetry of the face by using the mirror angle of the right side as a template. This can give a virtual image of the corrected defect that can be customised to fit the fractured region. This virtual image can then be converted to the file type used in Solid Freeform Fabrication machines and used to make a patient-tailored physical model of the defect.

Although the present invention is particularly applicable to scaffolds for tissue engineering it will be appreciated that the process can also be applied to other scaffolds and objects where intricate microporous structures are required, using appropriate polymers.

The following Examples further illustrate the present invention.

### EXAMPLE 1

Moulds were designed using a Model-Maker II. The design accounted for pore channel size and orientation for building and scaffolding purposes, and mould removal considerations. Prototype moulds were built using ProtoBuild with a 40 μm layer thickness to impart rigidity to the structure and produce smooth surface finishing and ProtoSupport. The support material was removed by a combination of temperature and ultrasonic agitation. The characteristics used were as follows:

Build layer: 0.0005 in. (0.013 mm) to 0.003 in. (0.076 mm)

Surface finish: 32-63 micro-inches (0.08-0.16 micrometres) (RMS)

Size of micro-droplet: 0.003 in. (0.076 mm)

Plotter carriage calibration: automatic, before each build cycle

Build envelope: X=12 in. (30.48 cm), Y=6 in. (15.24 cm), Z=8.5 in. (21.59 cm).

A 1% (weight/volume) dispersion of insoluble bovine collagen type I (Sigma-Aldrich, U.K) in 0.05M acetic acid was produced and homogenised using a conventional blender for 1 min. The dispersion of collagen was cast into the moulds and frozen in a freezer (approximate temperature of −20° C.) for 24 hours. The mould with frozen collagen was then immersed in propanone to dissolve the mould material. The remaining collagen sponge that was suspended in propanone was then critical point dried (Polaron Critical Point Drier) with carbon dioxide (CO₂). The morphology of the dry sponges was observed under a stereo-optical microscope or embedded in wax and then viewed under the stereo-optical microscope (Wild Heerbrugg, Leica). The embedding procedure involved placing the samples in molten wax at 65° C. under vacuum (<1 mbar) for 24 hours and then allowing the wax to solidify at room temperature for a further 24 hours. Similar results can be obtained using ethanol.

The presence of contamination from the mould materials was assessed by ultraviolet (UV) spectroscopy on collagen films. Films were cast from the collagen dispersion onto a flat glass surface and the solvent allowed to evaporate. The films were then immersed in a 0.5% weight/volume solution of ProtoBuild in ethanol for 10, 15 and 20 minutes, removed and allowed to air dry for 24 hours. UV spectroscopy in transmittance was performed on these collagen films and compared to control films.

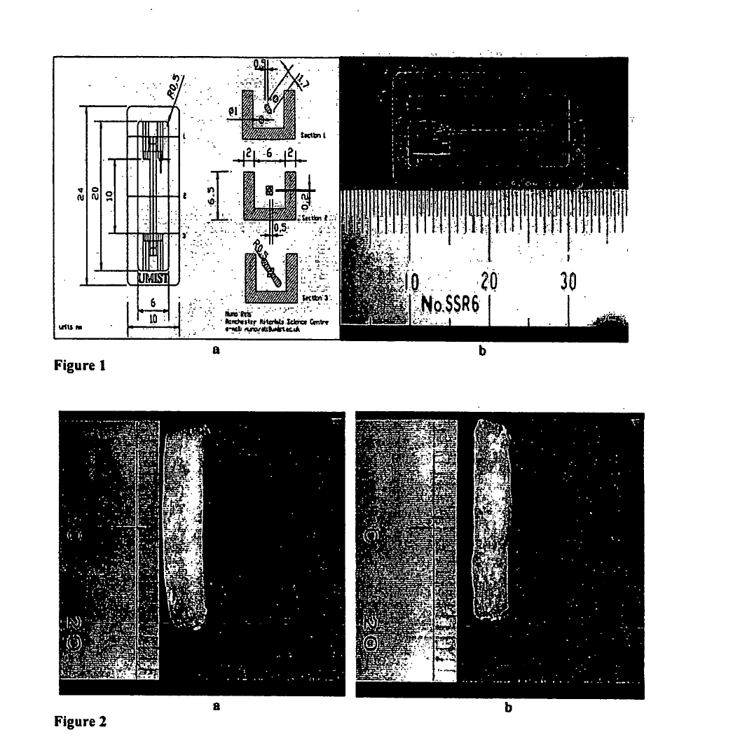

The results obtained are illustrated in the accompanying Figures in which: FIG. 1 (a) is a CAD sketch showing the dimensions of the mould while (b) is a photograph of the mould (units in mm).

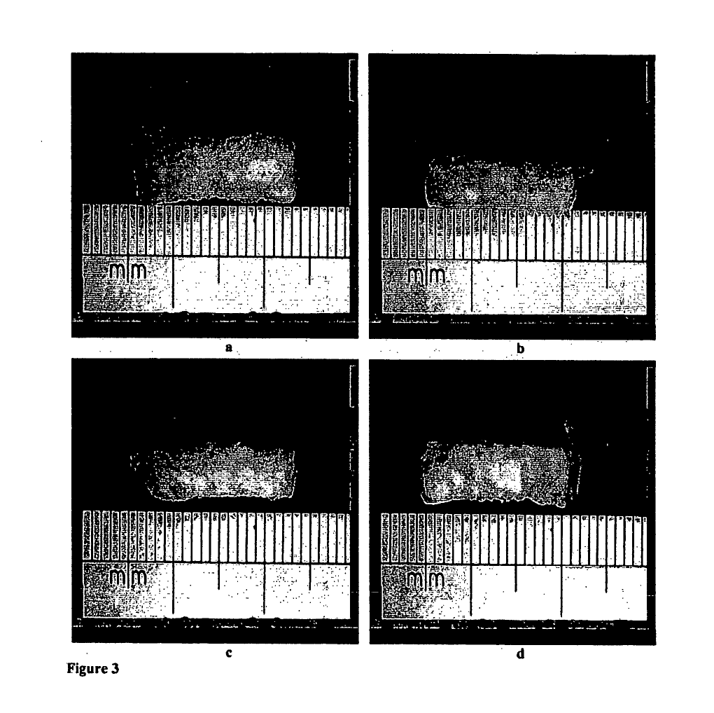

FIG. 2 shows top (a) and side (b) views of the collagen after immersion in propanone and the mould dissolved away. The box shaped structure has been retained and is an interconnected network of fibrils that is an inherent open cell structure.

FIG. 3 shows top (a), side (b), other side (c) and bottom (d) views of the scaffold after critical point drying; the general mould shape is present, but with some shrinkage.

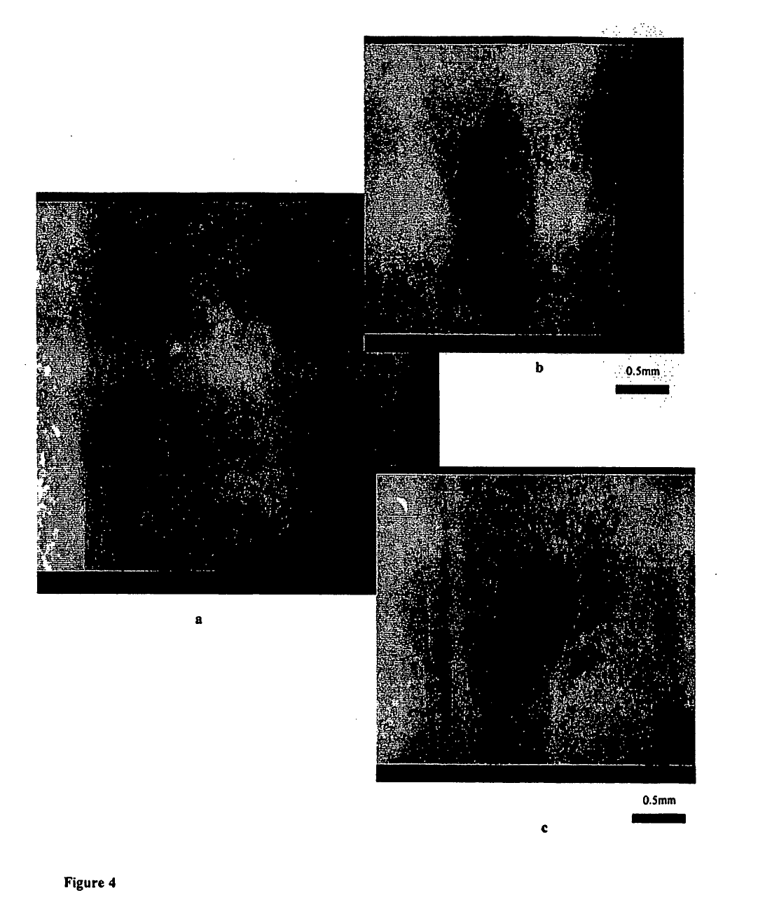

FIG. 4 shows the scaffold viewed from the edge. The inlet and outlet shafts shown in FIG. 1(a) are preserved with a well defined morphology. (b) shows the top right channel and (c) the bottom left channel. Originally 1 mm diameter they are now about 750 μm.

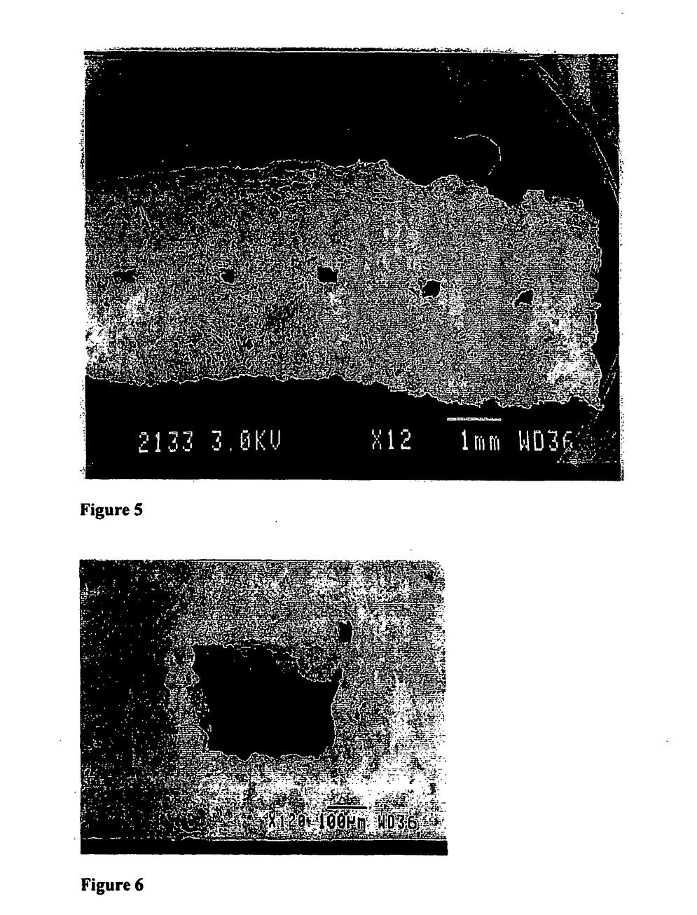

FIG. 5 is an SEM micrograph in the secondary electron mode of a section through another collagen scaffold made in accordance with this invention.

FIG. 6 is a view of the central channel of FIG. 5 at higher magnification. Note the well defined square shape.

### EXAMPLE 2

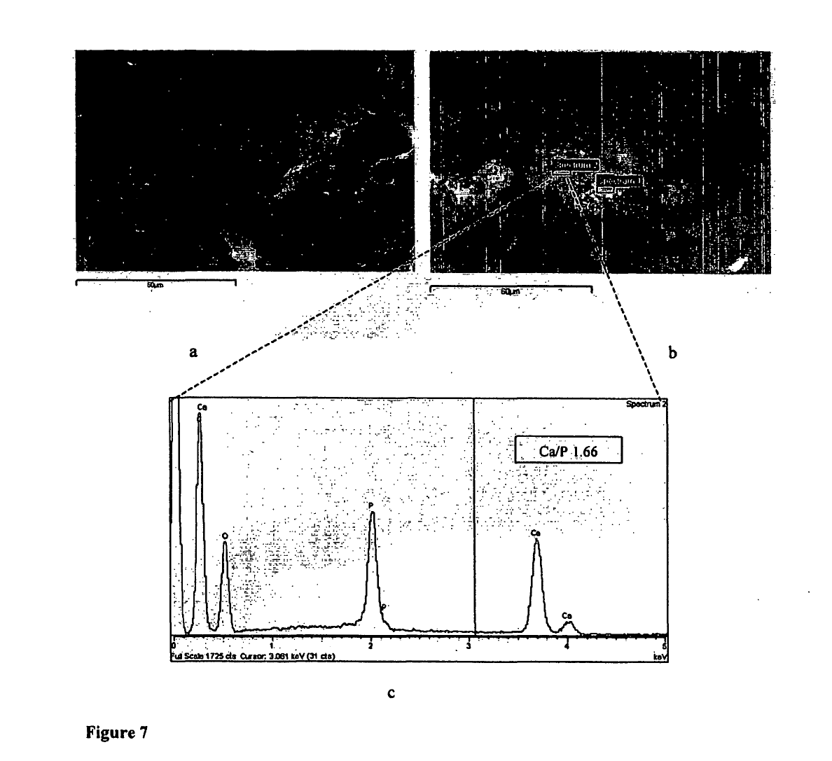

HA particles (Captal, Plasma Biotal Ltd) were mixed with collagen at a weight ratio of 2:1 in a dispersion in water. The HA/collagen dispersion was then cast into moulds made from phase change ink jet printing and frozen at −20° C. The mould was then removed by immersing the frozen HA/collagen-containing mould into ethanol, and the ethanol removed by critical point drying with liquid carbon dioxide. FIG. 7a shows a secondary electron micrograph of a composite scaffold obtained and FIG. 7b is the same area operated in the backscattered electron mode showing the brighter HA particles embedded in the collagen porous structure. Chemical analysis with a scanning electron microscope using energy dispersive X-ray spectroscopy (JSM-840A, JEOL, equipped with EDX detector) was performed on the HA to assess any changes to the calcium to phosphate ratio due to processing. The calcium to phosphate ratio of HA after undergoing processing varied between 1.47 and 1.66, values which are close to the stoichiometric constant of 1.67 for HA. FIG. 7c shows the calcium to phosphate ratio of processed HA in the area outlined in FIG. 7b.

1. A process for preparing a scaffold of biocompatible polymer which comprises placing a composition comprising the polymer in a mould possessing one or more voids therein, said mould being a negative of the desired shape of the scaffold, causing the polymer to acquire the shape of the mould and causing pores to be formed in the polymer, and removing the mould without affecting the polymer.

2. A process according to claim 1 wherein the polymer is biodegradable or bioresorbable.

3. A process according to claim 2 wherein the biodegradable polymer is collagen.

4. A process according to claim 3 wherein the biodegradable polymer is a mixture of collagen and elastin.

5. A process according to claim 1 wherein the scaffold also comprises a bio ceramic.

6. A process according to claim 1 wherein the mould is produced using solid freeform fabrication.

7. A process according to claim 6 wherein the mould is produced using phase change jet printing.

8. A process according to claim 1 wherein the polymer is collagen which is introduced into the mould as a dispersion in water having a concentration from 0.01 to 10% weight/volume.

9. A process according to claim 8 wherein the concentration of the collagen in the mould is increased by applying a removable absorbent for water to collagen dispersion in the mould.

10. A process according to claim 1 wherein electrical or magnetic particles are grafted onto the polymer before the composition is placed in the mould and an electrical or magnetic field, respectively, is applied to the composition in the mould to orient the polymer particles therein.

11. A process according to claim 10 wherein the particles are electrical and electrical particles are also applied to the mould.

12. A process according to claim 1 wherein electrical or magnetic particles are applied to the mould.

13. A process according to claim 1 wherein the composition is frozen while in the mould to acquire the shape of the mould.

14. A process according to claim 13 wherein the polymer is collagen and is frozen to a temperature from−20 C.to−80 C.

15. A process according to claim 1 wherein the mould is removed by the addition of a solvent therefore which is a non solvent for the biodegradable polymer.

16. A process according to claim 15 wherein the mould is dissolved in a polar solvent which is a non solvent for collagen.

17. A process according to claim 16 wherein the polar solvent is ethanol, 2-propanol, propanone, water or an aqueous ethanolic solution.

18. A process according to claim 15 wherein the polymer is collagen and the solvent for the mould is removed from the collagen by critical point drying using liquid carbon dioxide.

19. A process according to claim 1 wherein the scaffold is provided with a laminated or mosaic structure, with layers or regions having different chemical compositions.

20. A process according to claim 1 wherein the mould is shaped such that the external shape of the scaffold has the gross shape of the organ for which it is to act as a replacement.

21. A process according to claim 1 wherein the scaffold comprises one or more conduits either for the growth of peripheral nerves, blood vessels, connective tissue and/or highly vascularised vital organs,and/or for the provision of nutrients for such growth.

22. A process according to claim 1 wherein the mould is made of cholesterol.

23. A process according to claim 1 wherein the mould is made with the aid of a support of polyethylene glycol.

24. (Cancelled) A process according to claim 1 substantially as described in either of the Examples.

25. A scaffold of biocompatible polymer whenever prepared by a process as claimed in claim 1.

26. A scaffold of biocompatible polymer obtainable by a process claimed in claim 1.
